(19) United States
(12) Patent Application Publication
(10) Pub. No.: US 2009/0222006 A1
(43) Pub. Date: Sep. 3, 2009

(54) EXTERNAL FIXATOR

(76) Inventor: David M. Allison, Western Australia (AU)

Correspondence Address: VIDAS, ARRETT & STEINKRAUS, P.A., SUITE 400, 6640 SHADY OAK ROAD, EDEN PRAIRIE, MN, 55344, US

(21) Appl. No.: 12/093,067

(22) PCT Filed: Nov. 11, 2006
(86) PCT No.: PCT/AU2006/001663

§ 371 Date: Oct. 21, 2008

(30) Foreign Application Priority Data
Nov. 10, 2005 (AU) 2005906239

(51) Int. Cl. A61B 17/00

(52) U.S. Cl. 606/54; 606/57

(57) ABSTRACT

An external fixator (10) for assisting healing of a fracture in a bone (18) comprises a securing portion (16) that defines a void; and an elongate bracket portion (12) extends from the securing portion. The bracket portion is arranged to engage one or more first percutaneous bone fasteners (20) locatable in the bone on a first side of the fracture. In use, one or more second percutaneous bone fasteners (26) are located in the bone on a second side of the fracture such that the second fasteners extend into the void of the securing portion. The void can be filled with a cement compound to secure the second bone fasteners in place with respect to the securing portion.

### FIELD OF THE INVENTION

The present invention relates to an external fixator for assisting healing of a fracture in a bone.

### BACKGROUND OF THE INVENTION

Fracture of the distal end of the radius bone near the wrist is a relatively common injury. This injury is often referred to as a Colles fracture. A simple Colles fracture can be treated by merely realigning and immobilizing the fracture. More complex fractures may require the use of orthopedic devices to realign and reduce the fracture.

If wrist joint surfaces are damaged and not correctly realigned, the fracture may heal with an uneven joint surface, which can lead to accelerated wear and subsequent degenerative changes.

There are many methods used to realign or reduce fractures, such as a Colles fracture. One such method is ligamentotaxis. Ligamentotaxis relies on the attachment of ligaments, periosteum and other soft tissue attachments to the bone fracture fragments. By placing these soft tissue connections under tension the fracture is realigned or reduced.

Similarly, there are many methods of holding a reduced fracture in place after it has been realigned. One such method is external fixation, in which a device—usually comprising pins, rods, screws and/or other metal components—is attached percutaneously to pieces of bone, such that the device bridges the fracture. This type of device is commonly known as an external fixator.

In some cases of a Colles fracture, the external fixator is attached to the radius bone proximal to the fracture and to the metacarpal bones in the hand distal to the fracture. In this way, the fracture and the wrist joint are held in a fixed position during the healing process. However, fixing a joint in position can lead to increased stiffness of the joint and is undesirable.

Alternatively, an external fixator can be arranged such that the pins are inserted into the distal fragments of, for example, the fractured radius bone. This is a more desirable solution since mobility of the joint can be maintained during the healing process. However, when the distal fragments are small it can be difficult to hold the fracture in alignment by externally placed pins. This is particularly so when only one or two pins can be applied to hold the distal fragments in place and there are constraints on the direction in which they can be applied.

### SUMMARY OF THE INVENTION

According to a first aspect of the present invention, there is provided an external fixator for assisting healing of a fracture in a bone, the external fixator comprising:
a securing portion that defines a void; and
an elongate bracket portion extending from the securing portion, the bracket portion arranged to engage one or more first percutaneous bone fasteners locatable in the bone on a first side of the fracture;
whereby, in use, one or more second percutaneous bone fasteners are located in the bone on a second side of the fracture such that the second fasteners extend into the void of the securing portion, and the void can be filled with a cement compound to secure the second bone fasteners in place with respect to the securing portion.

Preferably, the securing portion is in the form of a loop. More preferably, the loop is a closed loop.

Preferably, part of the bracket portion forms a part of the securing portion. More preferably, the securing portion comprises an arcuate strip attached at each end to the bracket portion. Even more preferably, the arcuate strip is continuous with the bracket portion.

Preferably, the bracket portion comprises a plate having one or more holes extending therethrough for receiving the first bone fasteners. More preferably, the holes are arranged to extend through the plate transverse to the elongate direction of the bracket portion.

Preferably, each of the holes is provided with a threaded hole for receiving a threaded fastener to fix the respective first bone fastener in place, wherein the threaded hole is arranged to intersect with the respective hole.

Preferably, the second percutaneous bone fasteners are pins or rods, such as Kirschner wires. The pins or rods may be provided with a threaded portion for engaging bone.

In one embodiment, the external fixator further comprises at least one distractor comprising:
a first coupling portion for coupling with the bracket portion;
a shaft attached to the first coupling portion; and
a pin clamp having a second coupling portion which engages the shaft such that the pin clamp is moveable along the length of the shaft, and one or more holes for receiving third percutaneous bone fasteners;
wherein, in use, the position of the pin clamp can be adjusted to provide distraction in the longitudinal direction of the shaft.

Preferably, the shaft is threaded and the second coupling portion has a threaded hole which engages the thread of the shaft such that rotation of the shaft causes movement of the pin clamp along the length of the shaft. Preferably the shaft is longitudinally fixed to the first coupling portion and axially rotatable relative to the first coupling portion.

Preferably, the first coupling portion has a projecting stud that is received in a complementary hole in the bracket portion to couple the distractor to the bracket portion.

Preferably, the complementary hole is one of a plurality of like complementary holes.

According to a second aspect of the present invention, there is provided a method for holding in place a fracture in a bone, the method comprising the steps of:
providing an external fixator according to the first aspect;
securing one or more first bone fasteners percutaneously in the bone on the first side of the fracture;
fastening the bracket portion to the first bone fasteners;
securing one or more second bone fasteners percutaneously in the bone on the second side of the fracture, such that the second bone fasteners extend through the void defined by the securing portion; and
filling the void with a cement compound and allowing the cement compound to set so as to secure the second bone fasteners in place with respect to the external fixator.

After the cement compound has set, exposed ends of the second bone fasteners may be trimmed if desired.

In an embodiment the method further comprises realigning or reducing the fracture by moving the bone on the second side of the fracture prior to allowing the cement compound to set. Preferably the realignment or reduction occurs prior to filling the void with the cement compound. Even more preferably the realignment or reduction occurs prior to securing the second bone fasteners in the bone.

Preferably, the step of realigning or reducing the fracture further comprises providing one or more distractors, and for each distractor:
securing one or more third percutaneous bone fasteners to a piece of bone in the direction to be distracted;
securing a clamp of the distractor to the third bone fasteners;
attaching a coupling portion of the distractor to the bracket portion; and
adjusting the position of the clamp with respect to the bracket portion to attain the desired distraction and realignment or reduction of the fracture.

Preferably, once the cement compound is set the distractors are removed from the bracket portion and the third bone fasteners removed.

According to a third aspect of the present invention, there is provided an external fixator kit for assisting healing of a fracture in a bone, the external fixator kit comprising:
an external fixator comprising;
a securing portion that defines a void, and
an elongate bracket portion extending from the securing portion;
one or more first percutaneous bone fasteners locatable in the bone on a first side of the fracture and arranged to engage the bracket portion in use;
one or more second percutaneous bone fasteners locatable in the bone on a second side of the fracture such that the second fasteners extend into the void of the securing portion; and
a cement compound for filling the void so as to secure the second bone fasteners in place with respect to the securing portion.

According to a fourth aspect of the present invention, there is provided a distracter comprising:
an elongate bracket portion arranged to engage one or more first percutaneous bone fasteners locatable in bone on a first side of a bone fracture;
a first coupling portion for coupling with the bracket portion;
a shaft attached to the first coupling portion; and
a pin clamp having a second coupling portion which engages the shaft such that the pin clamp is moveable along the length of the shaft, and one or more holes for receiving further percutaneous bone fasteners locatable in bone on a second side of the bone fracture;
wherein, in use, the position of the pin clamp can be adjusted to provide distraction in the longitudinal direction of the shaft.

### BRIEF DESCRIPTION OF THE DRAWINGS

In order that the invention may be more easily understood, embodiments will now be described, by way of example only, with reference to the accompanying drawings, in which:

FIG. 1 is a schematic plan view of an external fixator according to a first embodiment of the present invention;

FIG. 2 is a side view of the external fixator of FIG. 1;

FIG. 3 is a perspective view of an external fixator with a distractor according to a second embodiment of the present invention;

FIG. 4 is a side view of the distractor of FIG. 3; and

FIG. 5 is a perspective view of an external fixator according to a third embodiment of the present invention.

### DETAILED DESCRIPTION OF THE PREFERRED EMBODIMENTS

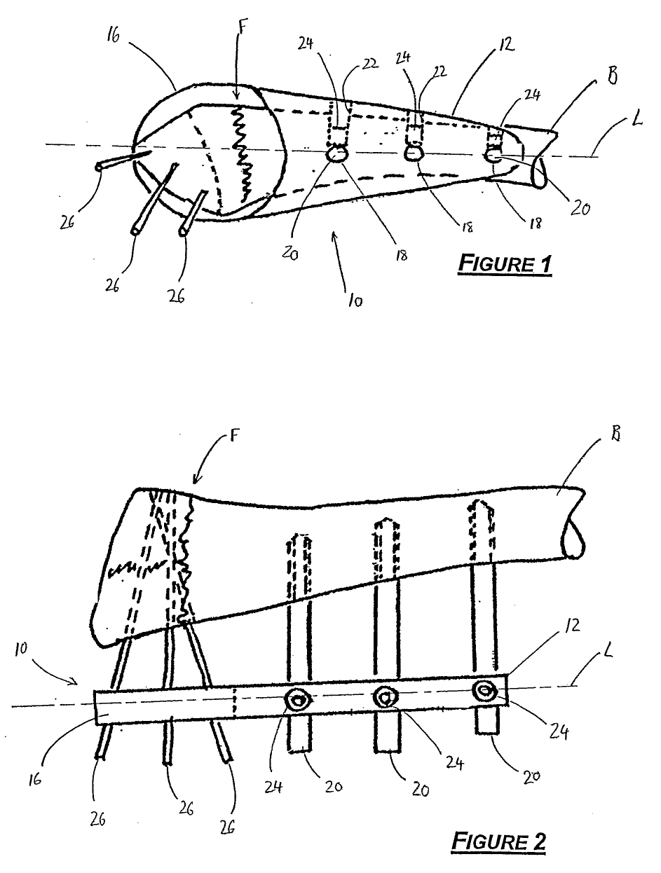

FIGS. 1 and 2 show an external fixator 10 according to a first embodiment of the present invention. The fixator 10 comprises a bracket portion 12 and a securing portion in the form of a closed loop 16 continuous with the bracket portion 12. The fixator 10 is shaped so as to extend along an elongate axis L. In use, the fixator 10 is attached to a bone B such that the elongate axis L is aligned generally with the direction of the bone B.

The bracket portion 12 is provided with three holes 18 which are arranged perpendicular to the elongate axis L. The holes 18 are arranged to receive one or more of a first type of bone fasteners, such as rods 20, in a clearance fit. Each hole 18 is provided with a corresponding hole 22 that extends through part of the bracket portion 12 and intersects with the side of the respective hole 18. The holes 22 are internally threaded to receive a grub screw 24.

The loop 16 is arranged so that a one or more of a second type of bone fasteners, such as Kirschner wires or thin pins 26, can be arranged to extend through the void on the inside of the loop 16 and into distal fragments of the bone B. As shown in the figures, the pins 26 can be arranged in many different orientations. A surgical cement compound, such as (poly)methylmethacrylate—also known as PMMA, is used to fill the volume of the void enclosed by the loop 16 and secure the pins 26 in position relative to the fixator 10.

In use, the surgeon has the freedom to select the number of pins 26 to be inserted into the bone fragments. In addition, the pins 26 can be oriented to suit the particular fracture, provided that the pins 26 extend through the loop 16.

FIGS. 1 and 2 show the fixator 10 applied to the distal end of a radius bone B across a fracture F. As mentioned above, the distal radius fracture is a common injury. The rods 20 are secured in the proximal portion of the bone B and the pins 26 are secured in fragments of the distal portion of the bone B.

In use, the rods 20 are secured percutaneously to the proximal portion of the bone B and the bracket portion 12 is then fastened to the rods 20. The location of the rods 20 is selected to correspond with the relative position of the holes 18 and such that the fixator 10 extends across the fracture F. The pins 26 are similarly secured percutaneously to the fragments of bone at the distal end of the bone B after the fracture is reduced. Clearly, the location and orientation of each pin 26, as well as the number of pins 26 used, will be selected for the particular fracture F. Finally, the cement compound (not shown) is used to fill the void enclosed by the loop 16 and secure the pins 26 in place relative to the fixator 10. Once setting of the cement has occurred, free ends of the pins 26 may be trimmed if desired.

The fixator 10 has been shown using rods 20 with a threaded portion that engages the bone B. However, it will be appreciated that alternate fixing methods may be used.

One advantage of the fixator according to the present invention is that the joint can remain active whilst the fracture is healing. Mobility during the healing process can help to reduce stiffness of the joint in the long term.

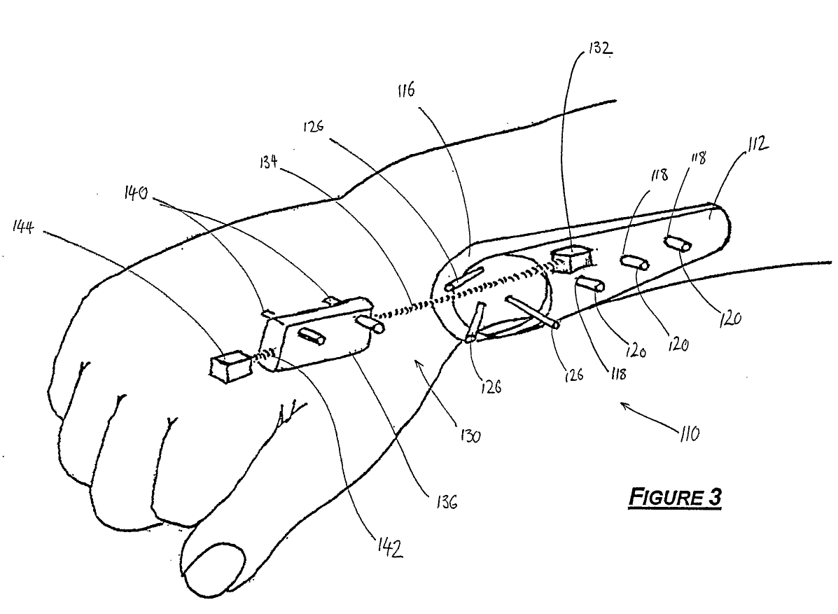

FIG. 3 shows an external fixator 110 according to a second embodiment of the present invention. The fixator 110 comprises a bracket portion 112 and a securing portion in the form of a loop 116 continuous with the bracket portion 112. The fixator 110 is shaped so as to extend along an elongate axis. In use, the fixator 110 is attached to a bone such that the elongate axis of the fixator 110 is aligned generally with the direction of the bone.

The bracket portion 112 is provided with three holes 118 which are arranged perpendicular to the elongate axis. The holes 118 are arranged to receive rods 120 in a clearance fit. Each hole 118 is provided with a corresponding hole that extends through part of the bracket portion 112 and opens into the side of the respective hole 118. The holes are internally threaded to receive a grub screw (not shown in FIG. 3).

The loop 116 is arranged so that a one or more of a second type of bone fasteners, such as Kirschner wires or thin pins 126, can be arranged to extend through the loop 116 and into distal fragments of the bone B. As shown in the figures, the pins 126 can be arranged in many different orientations. A surgical cement compound is used to fill the void enclosed by the loop 116 and set the pins 126 in position relative to the fixator 110.

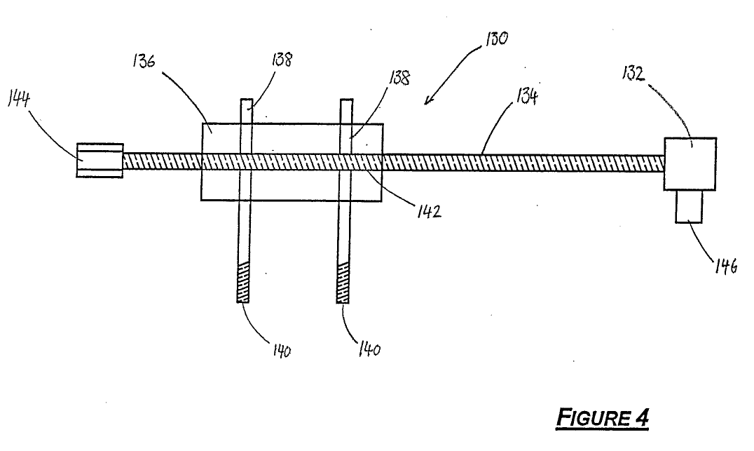

It has been shown that the principle of ligamentotaxis, which is obtained by longitudinal traction (or distraction), can be useful in realigning or reducing some fractures, such as distal radial fractures. To provide distraction, the fixator 110 further comprises a distractor 130, as shown in FIG. 4, which comprises a coupling portion 132, a threaded shaft 134 and a clamp, such as pin clamp 136. The pin clamp 136 is provided with two holes 138 for receiving distractor pins 140, and an internally threaded hole 142. The direction of the holes 138 and the direction of the threaded hole 142 are transverse. The threaded shaft 134 extends through the threaded hole 142.

The coupling portion 132 is attached to an end of the threaded shaft 134 in a swivel joint arrangement. Thus, the threaded shaft 134 can freely rotate with respect to the coupling portion 132. The opposite end of the threaded shaft 134 is provide with a head 144 that is shaped such that a tool can be used to effect rotation of the threaded shaft 134. The coupling portion 132 has a protruding stud 146 that engages a complementary hole (not shown) in the bracket portion 112.

In use, the distractor pins 140 can be applied percutaneously through the soft tissue of the hand into, for example, the second metacarpal bone. The pin clamp 136 of the distractor 130 is then attached to the exposed portions of the distractor pins 140. The coupling portion 132 is then attached to the bracket portion 112. The position of the pin clamp 136 along the threaded shaft 134 and relative to the fixator 110 is adjusted by rotation of the threaded shaft 134 until the desired longitudinal distraction is attained and the fracture reduced. Subsequently, the pins are placed through the void and across the fracture and the cement compound is applied to fill the void defined by the loop 116. Once the cement compound has set, the distractor 130 is removed from the bracket portion 112.

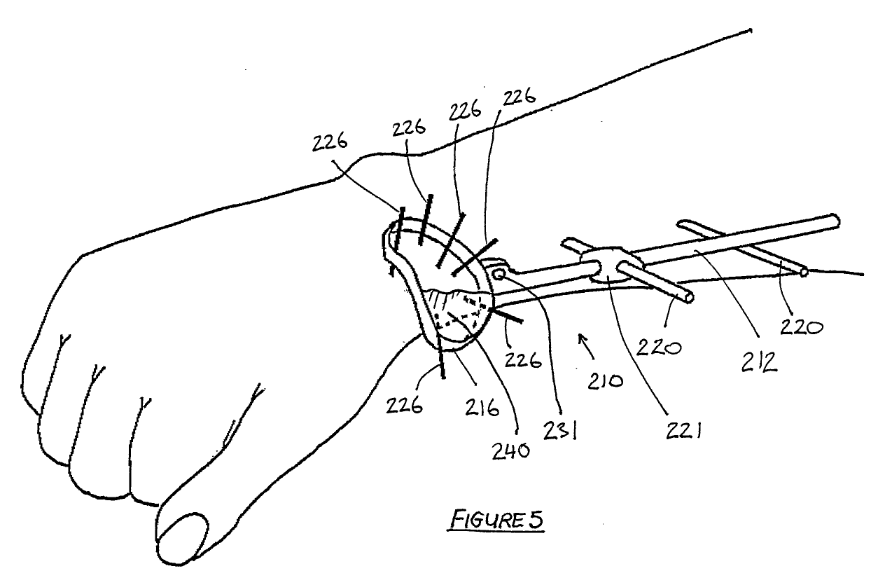

FIG. 5 shows an external fixator 210 according to a third embodiment in the present invention. The fixator 210 comprises a bracket portion 212 and a securing portion in the form of a loop 216 connected to the bracket portion 212. In this embodiment the bracket portion 212 is an elongate rod which in use is arranged so that its length is generally aligned with the direction of a bone in need of healing.

The bracket portion 212 is provided with a clamp 221 which receives a percutaneous bone fastener 220. The second bone fastener 220 shown is also clamped to the bracket portion 212 but for clarity purposes the clamp is not shown. The bracket portion 212 is provided with a hole 231 adjacent to the loop 216 for receiving protruding stud 146 of a distractor 136 should it need to be used. In this embodiment the loop 216 is generally kidney shaped. It can be seen that six percutaneous bone fastening pins 226 are used. The loop 216 is shown with cement 240 securing the pins 226 in place. The cement 240 is shown partially cut away.

It will be understood to persons skilled in the art of the invention that many modifications may be made without departing from the scope of the invention. For example, the fixator of the present invention may be provided with one or more holes in the bracket portion to receive the rods. Clearly, in use a surgeon may choose to use fewer rods than can be accommodated in the bracket portion, provided that the fixator is secured to the proximal portion of the bone.

The fixator may be further provided with a one or more membranes, mesh or similar on one or both sides of the loop 16. Accordingly, the void enclosed by the loop 16 and in particular the cement can be physically contained by the loop and the membranes or mesh. The membranes can be perforated to allow the pins to pass through the loop or the holes in the mesh perform this role. The cement can then be applied into the void defined by the loop and the membranes or mesh. The mesh or membrane(s) can assist to hold the pins in place while the cement sets. The holes in the mesh or markings on the membrane(s) can operate as a positioning guide.

While the closed loop 16 in the figures is shown to be generally circular, it will be appreciated that the loop can be provided in a number of shapes. For example, tear drop shaped.

The fixator is shown in FIGS. 1 to 3 as comprising a bracket portion in the form of a narrow plate. Furthermore, the loop is shown as being an arcuate strip joined at each end to the bracket portion, such that a closed loop is formed. It will be appreciated that alternative embodiments of the fixator may be provided, in which, for example, the bracket portion is in the form of a wire frame, a rod or a rigid tube of suitable material that is rigidly attached to a securing portion in the form of a ring. In this alternative embodiment, conventional pin/rod clamps may be used to secure the rods to the wire frame, rod or rigid tube.

The securing portion need not be a closed loop. The loop may be formed by two discontinuous arms, such that an opening is provided in the loop. In an embodiment in which the arms are made of a deformable material, the size of the void defined by the loop can be increased or decreased as desired. In use, any gap between the arms can be filled with the cement compound.

It will be appreciated that many alternative cement products may be used, such as, for example, a Calcium Phosphate (CaP) cement compound. The cement should be substantially inflexible once set.

It is recognized that ligamentotaxis in two (or more) planes can be of benefit in the reduction or realignment of a fracture. Accordingly, it will be appreciated that a plurality of distractors 130 may be used to achieve distraction in specific directions.

The coupling portion 132 has been shown as having a stud which is received in a hole in the bracket portion. However, it will be appreciated that alternative coupling arrangements can be employed. For example, the coupling portion 132 could be secured to the bracket portion 112 by a clamp.

The shaft 134 is in threaded engagement with threaded hole 142, however it will be appreciated that other mechanisms can be employed to move the pin clamp 136 along the length of the shaft 134, such as a rack and pinion.

While embodiments of the fixator have been described in connection with a fracture to the distal end of the radius bone, it will be appreciated that the fixator may be used to facilitate the healing of fractures in other bones of humans or animals.

Throughout this specification, except where the context requires otherwise due to express language or necessary implication, the words “comprise” or variations such as “comprises” or “comprising” are used in an inclusive sense, i.e. to specify the presence of the stated features but not to preclude the presence or addition of further features in various embodiments of the invention.

What is claimed is:

1. An external fixator for assisting healing of a fracture in a bone, the external fixator comprising:
a securing portion that defines a void; and
an elongate bracket portion extending from the securing portion, the bracket portion arranged to engage one or more first percutaneous bone fasteners locatable in the bone on a first side of the fracture;
whereby, in use, one or more second percutaneous bone fasteners are located in the bone on a second side of the fracture such that the second fasteners extend into the void of the securing portion, and the void can be filled with a cement compound to secure the second bone fasteners in place with respect to the securing portion.

2. An external fixator as claimed in claim 1, wherein the securing portion is in the form of a loop.

3. An external fixator as claimed in claim 1, wherein the loop is a closed loop.

4. An external fixator as claimed in claim 1, wherein part of the bracket portion forms a part of the securing portion.

5. An external fixator as claimed in claim 1, wherein the securing portion comprises an arcuate strip attached at each end to the bracket portion.

6. An external fixator as claimed in claim 1, wherein the arcuate strip is continuous with the bracket portion.

7. An external fixator as claimed in claim 1, wherein the bracket portion comprises a plate having one or more holes extending therethrough for receiving the first bone fasteners.

8. An external fixator as claimed in claim 7, wherein the holes are arranged to extend through the plate transverse to the elongate direction of the bracket portion.

9. An external fixator as claimed in claim 7 wherein each of the holes is provided with a threaded hole for receiving a threaded fastener to fix the respective first bone fastener in place, wherein the threaded hole is arranged to intersect with the respective hole.

10. An external fixator as in claim 1, wherein the second percutaneous bone fasteners are pins or rods.

11. An external fixator as claimed in claim 10, wherein the pins or rods are provided with a threaded portion for engaging bone.

12. An external fixator as in claim 1, wherein the second percutaneous bone fasteners are Kirschner wires.

13. An external fixator as in claim 1, further comprising at least one distractor comprising:
a first coupling portion for coupling with the bracket portion;
a shaft attached to the first coupling portion; and
a pin clamp having a second coupling portion which engages the shaft such that the pin clamp is moveable along the length of the shaft, and one or more holes for receiving third percutaneous bone fasteners;
wherein, in use, the position of the pin clamp can be adjusted to provide distraction in the longitudinal direction of the shaft.

14. An external fixator as claimed in claim 13, wherein the shaft is threaded and the second coupling portion has a threaded hole which engages the thread of the shaft such that rotation of the shaft causes movement of the pin clamp along the length of the shaft.

15. An external fixator as claimed in claim 14, wherein the shaft is longitudinally fixed to the first coupling portion and axially rotatable relative to the first coupling portion.

16. An external fixator as claimed in claim 13, wherein the first coupling portion has a projecting stud that is received in a complementary hole in the bracket portion to couple the distractor to the bracket portion.

17. An external fixator as claimed in claim 16, wherein the complementary hole is one of a plurality of like complementary holes.

18. A method for holding in place a fracture in a bone, the method comprising the steps of:
providing an external fixator according to the first aspect;
securing one or more first bone fasteners percutaneously in the bone on the first side of the fracture;
fastening the bracket portion to the first bone fasteners;
securing one or more second bone fasteners percutaneously in the bone on the second side of the fracture, such that the second bone fasteners extend through the void defined by the securing portion; and
filling the void with a cement compound and allowing the cement compound to set so as to secure the second bone fasteners in place with respect to the external fixator.

19. A method as claimed in claim 18, wherein the method further comprises realigning or reducing the fracture by moving the bone on the second side of the fracture prior to allowing the cement compound to set.

20. A method as claimed in claim 19, wherein the realignment or reduction occurs prior to filling the void with the cement compound.

21. A method as claimed in claim 19, wherein the realignment or reduction occurs prior to securing the second bone fasteners in the bone.

22. A method as claimed in claim 18, wherein after the cement compound has set, exposed ends of the second bone fasteners are trimmed.

23. A method as claimed in claim 19, wherein the step of realigning or reducing the fracture further comprises providing one or more distractors, and for each distractor:
securing one or more third percutaneous bone fasteners to a piece of bone in the direction to be distracted;
securing a clamp of the distractor to the third bone fasteners;
attaching a coupling portion of the distractor to the bracket portion; and
adjusting the position of the clamp with respect to the bracket portion to attain the desired distraction and realignment or reduction of the fracture.

24. A method as claimed in claim 23, wherein once the cement compound is set the distractors are removed from the bracket portion and the third bone fasteners removed.

25. An external fixator kit for assisting healing of a fracture in a bone, the external fixator kit comprising:
an external fixator comprising;
a securing portion that defines a void, and
an elongate bracket portion extending from the securing portion;
one or more first percutaneous bone fasteners locatable in the bone on a first side of the fracture and arranged to engage the bracket portion in use;
one or more second percutaneous bone fasteners locatable in the bone on a second side of the fracture such that the second fasteners extend into the void of the securing portion; and
a cement compound for filling the void so as to secure the second bone fasteners in place with respect to the securing portion.

26. (canceled)

27. (canceled)

28. (canceled)
